(19) United States
(12) Patent Application Publication
(10) Pub. No.: US 2009/0188812 A1
(43) Pub. Date: Jul. 30, 2009

(54) Cholesterol Sensor

(76) Inventors: John Morton Broughall, Oxford (GB); Herbert Frank Askew, Oxford (GB); Lindy Murphy, Oxford (GB)

Correspondence Address: QUARLES & BRADY LLP, 411 E. WISCONSIN AVENUE, SUITE 2040, MILWAUKEE, WI, 53202-4497, US

(21) Appl. No.: 12/298,876

(22) PCT Filed: May 14, 2007
(86) PCT No.: PCT/GB07/01769

§ 371 Date: Oct. 28, 2008

(30) Foreign Application Priority Data
May 12, 2006 (GB) 0609493.2

(51) Int. Cl. G01N 33/92; C12Q 1/60

(52) U.S. Cl. 205/777.5; 205/792; 204/403.14

(57) ABSTRACT

A method for the determination of the amount of cholesterol in lipoproteins other than high density lipoproteins in a lipoprotein-containing sample, said method comprising (a) electrochemically determining the amount of cholesterol bound to high density lipoproteins in the sample, (b) electrochemically determining the total amount of cholesterol in the sample, and subtracting the result of (a) from the result of (b).

### FIELD OF THE INVENTION

The present invention relates to a method for determining the amount of cholesterol bound to lipoproteins other than high density lipoproteins in a lipoprotein-containing sample. The invention also relates to a kit for use in such a method.

### BACKGROUND TO THE INVENTION

Cholesterol in plasma is contained in high density lipoprotein (HDL), low density lipoprotein (LDL), intermediate density lipoprotein (IDL), very low density lipoprotein (VLDL) and chylomicrons (CM). The cholesterol in HDL is often thought of as “good” cholesterol since HDL cholesterol content has been shown to reduce the risk of cardiac disease. On the other hand, the cholesterol in LDL is often thought of as “bad” cholesterol since high LDL cholesterol content has been shown to increase the risk of cardiac disease. Measurement of LDL cholesterol as an indicator of risk of cardiac disease is therefore recommended by the National Cholesterol Education Program 3^{rd} guidelines.

Two types of technique have typically been used in the past to determine LDL cholesterol levels. In one technique, measurements of the total cholesterol, HDL cholesterol and triglyceride content of a sample are made and the Friedwald equation used to calculate the LDL cholesterol content. This type of measurement, however, involves a triglyceride assay which can only be accurately carried out on a starved sample. Since such samples are not always available for analysis, it is not always possible to achieve reliable results in this manner. Techniques which do not require a starved sample are desired.

Alternative techniques aim to directly measure the LDL cholesterol content. Initial laboratory methods, adapted from research techniques, required a manual separation step, for example by ultracentrifugation, followed by analysis of the cholesterol content in each of the lipoprotein fractions individually. Such techniques, however, cannot be fully automated and require complex processing steps to be carried out. More recently, some techniques have been proposed which do not require prior separation of the various lipoprotein fractions and enable the process to be automated. In one approach, certain surfactants are used which break down the various lipoprotein fractions at different rates. For example, a surfactant might initially react more quickly with HDL, and reaction with LDL might occur more slowly. By measurement of the cholesterol content at a given time after addition of the surfactant, a measurement having a greater dependency on the LDL cholesterol content than on the HDL cholesterol content can be made.

This latter approach, however, has not generated the required accuracy and reliability in its results and the measurement still retains some degree of dependency on the content of cholesterol in HDL, IDL, VLDL and CM. A different approach is therefore required which provides a simple and yet reliable and accurate indicator of the risk of cardiac disease.

### SUMMARY OF THE INVENTION

The present invention uses an alternative approach, which relies on the measurement of the total cholesterol and HDL cholesterol contents of a sample only. Thus, the technique does not require measurement of the triglyceride content and a starved sample is not required. In the present invention, the non-HDL cholesterol content (i.e. the cholesterol content in lipoproteins other than HDL, namely LDL, IDL, VLDL and CM) is determined by subtracting the HDL cholesterol content from the total cholesterol content. This measurement has been found to provide a good correlation with the LDL-cholesterol content of a sample, and accordingly also with the risk of cardiac disease.

The present invention accordingly provides a method for the determination of the amount of cholesterol in lipoproteins other than high density lipoproteins in a lipoprotein-containing sample, said method comprising (a) electrochemically determining the amount of cholesterol bound to high density lipoproteins in the sample, (b) electrochemically determining the total amount of cholesterol in the sample, and subtracting the result of (a) from the result of (b).

By use of an electrochemical analysis, the present invention provides a particularly simple and rapid method for analysing the non-HDL cholesterol content. In particular, determinations (a) and (b) can be carried out simultaneously or substantially simultaneously, enabling the non-HDL cholesterol content to be determined in a matter of a minute or a few minutes from addition of a sample to a test device. Further, the test can be carried out by unskilled technicians and requires no specialist equipment. In one embodiment, the test can be carried out on a portable hand-held device which is appropriate for use in a medical environment, for example in a doctor's surgery, a hospital room or ward, or by the patient themselves at home.

The present invention also provides a kit for the determination of the amount of cholesterol in lipoproteins other than high density lipoproteins in a lipoprotein-containing sample, the kit comprising
a first electrochemical cell having a working electrode, a reference or pseudo reference electrode and optionally a separate counter electrode;
a first series of reagents for determining the HDL cholesterol content of the sample, said first series of reagents being associated with said first electrochemical cell;
a second electrochemical cell having a working electrode, a reference or pseudo reference electrode and optionally a separate counter electrode;
a second series of reagents for determining the total cholesterol content of the sample, said second series of reagents being associated with said second electrochemical cell;
a power supply for applying a potential across each cell;
a measuring instrument for measuring the resulting electrochemical response of each cell; and
a calculator for calculating the amount of cholesterol in lipoproteins other than high density lipoproteins.

Also provided is a method of operating the kit of the invention, said method comprising
(i) contacting a sample with the first series of reagents, such that the resulting mixture of sample and first series of reagents is in contact with the working electrode of the first electrochemical cell;
(ii) contacting the sample with the second series of reagents, such that the resulting mixture of sample and second series of reagents is in contact with the working electrode of the second electrochemical cell;
(iii) applying a potential across each electrochemical cell;
(iv) electrochemically detecting the amount of product formed in each cell by measuring the resulting electrochemical response; and
(v) calculating the amount of cholesterol bound to non-HDL lipoproteins in the sample.

### BRIEF DESCRIPTION OF THE FIGURES

FIG. 1 depicts an electrochemical cell of a device according to the invention.

FIG. 2 depicts an electrochemical sensor strip of a device according to the invention.

FIG. 3 depicts the results of a test protocol to determine whether the surfactant Amphitol 20N selectively breaks down HDL.

FIGS. 4a and b plot the results of total cholesterol tests carried out using donor plasma samples.

FIGS. 5a and b plot the results of HDL cholesterol tests carried out using donor plasma samples.

FIGS. 6a and b plot the correlation between the non-HDL cholesterol content of plasma samples measured in accordance with the invention, and the LDL cholesterol content of the samples measured by a Randox SPACE clinical analyser.

FIG. 7 shows the correlation between the non-HDL cholesterol content of plasma samples measured in accordance with the invention and the LDL cholesterol content of the samples determined using the Friedwald equation.

FIGS. 8(a) and (b) depict calibration plots (I_{OX} (nA) versus concentration (mM)) for HDL and TC respectively for sensors produced in accordance with Example 3.

FIGS. 9 to 11 show the relationship between the LDL content as measured using the reference method and that determined from the TC and HDL measurements made using the sensors of Examples 3 to 5.

FIGS. 12(a) and (b) depict calibration plots (I_{OX} (nA) versus concentration (mM)) for HDL and TC for the sensors of Example 7.

FIG. 13 shows the relationship between the LDL content as measured by the Randox analyser and that calculated according to the invention using the TC and HDL measurements made using the sensors of Example 7.

### DETAILED DESCRIPTION OF THE INVENTION

The method of the present invention involves carrying out a test for the HDL cholesterol content of a sample as well as the total cholesterol content of a sample and determining the non-HDL cholesterol content by simple subtraction. This very simple technique has been found to have a good correlation with the LDL cholesterol content of a sample, and therefore with risk of cardiac disease.

The HDL cholesterol test may be carried out by any electrochemical method. An electrochemical method is one in which the reaction of cholesterol is detected by measuring the electrochemical response at an electrode. Typically, a selective reagent is added to ensure that during the timeframe of measurement only the HDL cholesterol is able to react. The sample is subsequently reacted with a cholesterol ester hydrolysing reagent and either cholesterol oxidase or cholesterol dehydrogenase. The amount of cholesterol which has reacted with the cholesterol oxidase or cholesterol dehydrogenase is determined by measuring the electrochemical response at an electrode.

The selective reagent may be any material which enables HDL cholesterol to react with the cholesterol ester hydrolysing reagent and cholesterol oxidase or dehydrogenase. Suitable selective reagents include complexing agents, which form a complex with lipoproteins other than HDL. Examples of complexing agents include polyanions, combinations of polyanions with divalent metal salts, and antibodies capable of binding to apoB containing lipoproteins. The polyanions may be selected from phosphotungstic acid and salts thereof, dextran sulphuric acid and salts thereof, polyethylene glycol and heparin and salts thereof. Once in complexed form, the lipoproteins other than HDL are unavailable for reaction and therefore do not interfere with the cholesterol measurement.

In a preferred embodiment, selectivity is achieved by reacting the sample with a specific surfactant which is highly selective for HDL over LDL, VLDL and CM. Thus, the surfactant makes available for measurement the cholesterol and cholesterol esters bound to HDL, whilst those bound to LDL, VLDL and CM remain bound to the lipoprotein structure and substantially do not react in the later measurement of the cholesterol content.

The surfactants employed in this embodiment are those which are believed to selectively break down high density lipoproteins in a sample. The invention is not however, limited to this mode of action. The surfactants are therefore those which selectively enable HDL cholesterol to react in a cholesterol assay, whilst LDL cholesterol is substantially unable to react. This means that the surfactant reacts preferentially with HDL compared with LDL, VLDL and CM. In the context of the present invention, a surfactant which selectively breaks down HDL, or a surfactant which selectively enables HDL cholesterol to react in a cholesterol assay, is typically a surfactant having a differentiation between HDL and LDL of at least 50%, preferably at least 60%, at least 70%, at least 80% or most preferably at least 90%.

The differentiation between HDL and LDL can be determined according to the equation (i):

$$Differentiation ( \% ) = \frac{{G}_{HDL} - {G}_{LDL}}{{G}_{HDL}} \times 100 \qquad ( i )$$

wherein Gₓ is the gradient of the measured response to X (e.g. measured current vs the known concentration of X). The measured response may be any parameter which relates (or corresponds) to the lipoprotein concentration e.g. it may be a parameter which is proportional to the concentration.

The skilled person can therefore easily determine whether any given surfactant is one which selectively breaks down HDL by using the chosen surfactant to measure the HDL cholesterol content of a sample of known HDL cholesterol content (and which does not contain LDL), and correspondingly measuring the LDL cholesterol content of a sample of known LDL cholesterol content (and which does not contain HDL) using the same procedure. The differentiation value can be calculated from the results. An example of the procedure for measuring cholesterol contents using the surfactant Amphitol 20N is given in Example 1.

In the present invention, the concentration of HDL is measured electrochemically, typically by determining the current generated at an electrode on electrochemical conversion of cholesterol to cholestenone. The measured current value is therefore typically used to determine the gradient.

In a preferred embodiment, the selective surfactants of the invention substantially do not break down LDL. Therefore, the differentiation between HDL and LDL is constant over time. However, some surfactants may still break down LDL, albeit very slowly. In this case, the differentiation between HDL and LDL may vary over time. The differentiation should be measured using a time lapse between addition of reagents to the sample and measurement of the cholesterol content, which is the same as the time lapse to be used during HDL cholesterol testing. Such time lapse is typically in the order of 3 minutes or less, preferably 120 seconds or less, 90 seconds or less or 60 seconds or less. In the context of the invention, a selective surfactant is typically a surfactant having a differentiation between HDL and LDL of at least 50%, preferably at least 60%, at least 70%, at least 80% or most preferably at least 90%, when measuring the cholesterol contents using the procedure described in Example 1 and a time lapse between addition of reagents to the sample and measurement of 62 seconds.

Examples of surfactants which preferentially react with HDL include non-ionic surfactants such as polyoxyalkylene derivatives, perfluorinated alkanes or perfluorinated alkyl-group containing compounds, sucrose esters, tetramethyldecynediol and ethoxylated tetramethyldecynediols, polyalkylene oxide modified polydimethylsiloxane which is optionally combined with polyalkylene oxide, isononylphenoxypoly(glycidol), hydroxyethylglucamide derivatives, N-methyl-N-acyl-glucamine derivatives, maltosides and thiomaltosides; as well as amphoteric surfactants such as alkylbetaine derivatives, alkylamine oxides and ammonioalkylsulfonates.

In one embodiment of the invention, the surfactants are selected from non-ionic surfactants such as polyoxyalkylene derivatives, perfluorinated alkanes or perfluorinated alkyl-group containing compounds, sucrose esters, tetramethyldecynediol and ethoxylated tetramethyldecynediols, polyalkylene oxide modified polydimethylsiloxane which is optionally combined with polyalkylene oxide, isononylphenoxypoly(glycidol) and hydroxyethylglucamide derivatives, as well as amphoteric surfactants such as alkylbetaine derivatives, alkylamine oxides and ammonioalkysulfonates.

Preferred surfactants for use in the HDL assay of the present invention include polyoxyethylene lauryl ether, Emulgen 109P (Kao Corporation), Emulgen 1135S-70 (Kao Corporation), polyoxyalkylene distyrenated phenyl ether (Emulgen A-90, Kao Corporation), polyoxyalkylene allylphenyl ether (Newkalgen FS12, Takemoto Oil & Fat Co. Ltd), p-isononylphenoxypoly(glycidol) (Surfactant 10G, Surfactant Tool Kit, Research Diagnostics Inc.), Silwet L-7600 (Surfactant Tool Kit, Research Diagnostics Inc.), PEG-30 tetramethyl decynediol (Surfynol 485, Surfactant Tool Kit, Research Diagnostics Inc.), sucrose monocaprate (Sigma Aldrich Co. Ltd), perfluoro C₆-C₁₆ alkanes (Zonyl FSN100, Surfactant Tool Kit, Research Diagnostics Inc.), HEGA-8, HEGA-9, HEGA-10, C-HEGA-9, C-HEGA-10, n-octyl-N,N-dimethyl-3-ammonio-1-propanesulfonate (Zwittergent 3-08, Calbiochem), lauryl dimethyl amine oxide (Amphitol 20N, Kao Corporation), cocamidopropylbetaine (Softazoline CPB, Kawaken Fine Chemicals Co. Ltd) and lauramidopropylbetaine (Softazoline LPB-R, Kawaken Fine Chemicals Co. Ltd).

In one embodiment of the invention, preferred surfactants for use in the HDL assay Emulgen 1135S-70 (Kao Corporation), polyoxyalkylene allylphenyl ether (Newkalgen FS12, Takemoto Oil & Fat Co. Ltd), p-isononylphenoxypoly(glycidol) (Surfactant 10G, Surfactant Tool Kit, Research Diagnostics Inc.), Silwet L-7600 (Surfactant Tool Kit, Research Diagnostics Inc.), PEG-30 tetramethyl decynediol (Surfynol 485, Surfactant Tool Kit, Research Diagnostics Inc.), sucrose monocaprate, (Sigma Aldrich Co. Ltd), perfluoro C₆-C₁₆ alkanes (Zonyl FSN100, Surfactant Tool Kit, Research Diagnostics Inc.), HEGA-8, HEGA-9, HEGA-10, C-HEGA-9, C-HEGA-10, n-octyl-N,N-dimethyl-3-ammonio-1-propanesulfonate (Zwittergent 3-08, Calbiochem), lauryl dimethyl amine oxide (Amphitol 20N, Kao Corporation), cocamidopropylbetaine (Softazoline CPB, Kawaken Fine Chemicals Co. Ltd) and lauramidopropylbetaine (Softazoline LPB-R, Kawaken Fine Chemicals Co. Ltd).

In a further embodiment, preferred surfactants for use in the HDL assay include Emulgen 1135S-70 (Kao Corporation), p-isononylphenoxypoly(glycidol) (Surfactant 10G, Surfactant Tool Kit, Research Diagnostics Inc.), Silwet L-7600 (Surfactant Tool Kit, Research Diagnostics Inc.), PEG-30 tetramethyl decynediol (Surfynol 485, Surfactant Tool Kit, Research Diagnostics Inc.), sucrose monocaprate, perfluoro C₆-C₁₆ alkanes (Zonyl FSN100, Surfactant Tool Kit, Research Diagnostics Inc.), HEGA-8, HEGA-9, HEGA-10, C-HEGA-9, C-HEGA-10, n-octyl-N,N-dimethyl-3-ammonio-1-propanesulfonate (Zwittergent 3-08, Calbiochem), lauryl dimethyl amine oxide (Amphitol 20N, Kao Corporation), cocamidopropylbetaine (Softazoline CPB, Kawaken Fine Chemicals Co. Ltd) and lauramidopropylbetaine (Softazoline LPB-R, Kawaken Fine Chemicals Co. Ltd). Emulgen 1135S-70 (Kao Corporation) and hydroxyethyl glucamide derivatives including HEGA-8, HEGA-10, C-HEGA-9, C-HEGA-10 are particularly preferred. Sucrose monocaprate is also preferred.

In one embodiment of the invention, preferred surfactants are sucrose esters, maltosides. Particularly preferred surfactants are sucrose monocaprate, sucrose mono decanoate, n-octyl-β-D-maltoside, n-decyl-β-D-maltoside Cymal-4 and Cymal-5.

In an alternative embodiment, preferred surfactants are hydroxyethyl glucamide derivatives and N-acyl-N-methyl glucamine derivatives. Particularly preferred surfactants are HEGA-8, HEGA-10, HEGA-9, C-HEGA-9, C-HEGA-10 and MEGA-8.

Surfactants may be used singly or two or more different surfactants may be used in combination. The total amount of surfactant used is typically up to 200 mg per ml of sample to be tested, preferably up to 100 mg/ml, for example about 50 mg/ml.

In addition to the selective surfactant or other selective reagent, the sample is typically reacted with a cholesterol ester hydrolysing reagent and cholesterol oxidase or cholesterol dehydrogenase. The cholesterol contained in HDL lipoproteins may be in the form of free cholesterol or cholesterol esters. The cholesterol ester hydrolysing reagent is therefore used to break down any cholesterol esters into free cholesterol. Any reagent capable of hydrolysing cholesterol esters to cholesterol may be used. The reagent should be one which does not interfere with the reaction of cholesterol with cholesterol oxidase or cholesterol dehydrogenase and any subsequent steps in the assay. Preferred cholesterol ester hydrolysing reagents are enzymes, for example cholesterol esterase and lipases. A suitable lipase is, for example, a lipase from a Pseudomonas or Chromobacterium viscosum species. Commercially available enzymes, optionally containing additives such as stabilisers or preservatives may be used, e.g. those available from Toyobo or Amano. The cholesterol ester hydrolysing reagent may be used in an amount of from 0.1 to 20 mg per ml of sample, preferably from 0.5 to 15 mg per ml.

Any commercially available forms of cholesterol oxidase and cholesterol dehydrogenase may be employed. For instance, the cholesterol dehydrogenase is, for example, from the Nocardia species. The cholesterol oxidase or cholesterol dehydrogenase may be used in an amount of from 0.01 mg to 100 mg per ml of reagent mixture. In one embodiment, the cholesterol oxidase or dehydrogenase is used in an amount of from 0.1 to 80 mg per ml of sample, preferably from 0.5 to 30 mg per ml.

Each of the enzymes may contain additives such as stabilisers or preservatives. Further, each of the enzymes may be chemically modified.

The surfactant or other selective reagent may be added to the sample prior to addition of the other reagents or simultaneously with the addition of the other reagents. In a preferred embodiment, cholesterol ester hydrolysing reagent, cholesterol oxidase or dehydrogenase and a selective surfactant are present in a single reagent mixture which is combined with the sample in a single step. In a particularly preferred embodiment, the method involves a single step of contacting the sample with reagents, so that only a single reagent mixture need be provided. The reagent mixture of the invention typically comprises cholesterol ester hydrolysing reagent in an amount of from 0.1 to 25 mg, e.g. from 0.1 to 20 mg, preferably from 0.5 to 10 mg per ml of sample and cholesterol dehydrogenase in an amount of from 0.1 to 80 mg, preferably from 0.5 to 25 mg per ml of sample. A selective reagent is also present, typically a selective surfactant in an amount of up to 50 mg, preferably up to 20 mg, for example about 5 mg per ml of sample.

In order to detect the reaction of the cholesterol oxidase or cholesterol dehydrogenase at an electrode, the sample is typically also reacted with a coenzyme capable of interacting with cholesterol oxidase or cholesterol dehydrogenase, and a redox agent which is capable of being oxidised or reduced to form a product which can be electrochemically detected at an electrode. The mixture of sample and reagents is contacted with a working electrode of an electrochemical cell so that redox reactions occurring can be detected. A potential is applied across the cell and the resulting electrochemical response, typically the current, is measured.

In this preferred embodiment, the amount of HDL-cholesterol is measured in accordance with the following assay:

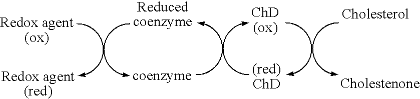

where ChD is cholesterol dehydrogenase. Cholesterol dehydrogenase could be replaced with cholesterol oxidase in this assay if desired. The amount of reduced redox agent produced by the assay is detected electrochemically. Additional reagents may also be included in this assay if appropriate.

Typically the coenzyme is NAD⁺ or an analogue thereof. An analogue of NAD⁺ is a compound having structural characteristics in common with NAD⁺ and which also acts as a coenzyme for cholesterol dehydrogenase. Examples of NAD⁺ analogues include APAD (Acetyl pyridine adenine dinucleotide); TNAD (Thio-NAD); AHD (acetyl pyridine hypoxanthine dinucleotide); NaAD (nicotinic acid adenine dinucleotide); NHD (nicotinamide hypoxanthine dinucleotide); and NGD (nicotinamide guanine dinucleotide). The coenzyme is typically present in the reagent mixture in an amount of from 1 to 20 mM, for example from 3 to 15 mM, preferably from 5 to 10 mM.

Typically, the redox agent should be one which can be reduced in accordance with the assay shown above. In this case, the redox agent should be one which is capable of accepting electrons from a coenzyme (or from a reductase as described below) and transferring the electrons to an electrode. The redox agent may be a molecule or an ionic complex. It may be a naturally occurring electron acceptor such as a protein or may be a synthetic molecule. The redox agent will typically have at least two oxidation states.

Preferably, the redox agent is an inorganic complex. The agent may comprise a metallic ion and will preferably have at least two valencies. In particular, the agent may comprise a transition metal ion and preferred transition metal ions include those of cobalt, copper, iron, chromium, manganese, nickel, osmium or ruthenium, for example, cobalt, copper, iron, chromium, manganese, nickel or ruthenium. The redox agent may be charged, for example it may be cationic or alternatively anionic. An example of a suitable cationic agent is a ruthenium complex such as Ru(NH₃)₆³⁺, an example of a suitable anionic agent is a ferricyanide complex such as Fe(CN)₆³⁻. Examples of complexes which may be used include Cu(EDTA)²⁻, Fe(CN)₆³⁻, Fe(CN)₅(O₂CR)³⁻, Fe(CN)₄(oxalate)³⁻, Ru(NH₃)₆³⁺, Ru(acac)₂(Py-3-CO₂H)(Py-3-CO₂) chelating amine ligand derivatives thereof (such as ethylenediamine), Ru(NH₃)₅(py)³⁺, ferrocenium and derivatives thereof with one or more of groups such as —NH₂, —NHR, —NHC(O)R, and —CO₂H substituted into one or both of the two cyclopentadienyl rings. Preferably the inorganic complex is Fe(CN)₆³⁻, Ru(NH₃)₆³⁺, or ferrocenium monocarboxylic acid (FMCA). Ru(NH₃)₆³⁺ and Ru(acac)₂(Py-3-CO₂H)(Py-3-CO₂) are preferred.

The redox agent is typically present in the reagent mixture in an amount of from 10 to 200 mM, for example from 20 to 150 mM, preferably from 30 to 100 mM or up to 80 mM.

In a preferred embodiment, the reagent mixture used in the electrochemical assay additionally comprises a reductase. The reductase typically transfers two electrons from the reduced NAD and transfers two electrons to the redox agent. The use of a reductase therefore provides swift electron transfer.

Examples of reductases which can be used include diaphorase and cytochrome P450 reductases, in particular, the putidaredoxin reductase of the cytochrome P450_{cam} enzyme system from Pseudomonas putida, the flavin (FAD/FMN) domain of the P450_{BM-3} enzyme from Bacillus megaterium, spinach ferrodoxin reductase, rubredoxin reductase, adrenodoxin reductase, nitrate reductase, cytochrome b₅ reductase, corn nitrate reductase, terpredoxin reductase and yeast, rat, rabbit and human NADPH cytochrome P450 reductases. Preferred reductases for use in the present invention include diaphorase and putidaredoxin reductases.

The reductase may be a recombinant protein or a naturally occurring protein which has been purified or isolated. The reductase may have been mutated to improve its performance such as to optimise the speed at which it carries out the electron transfer or its substrate specificity.

The reductase is typically present in the reagent mixture in an amount of from 0.5 to 100 mg/ml, for example from 1 to 50 mg/ml, 1 to 30 mg/ml or from 2 to 20 mg/ml.

In a preferred embodiment of the invention, the general scheme of the electrochemical assay is as follows:

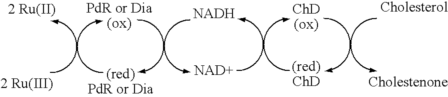

### Where

PdR—is putidaredoxin reductase
Dia—is diaphorase
ChD—is cholesterol dehydrogenase.

The reagent mixture optionally contains one or more additional components, for example excipients and/or buffers and/or stabilisers. Excipients are preferably included in the reagent mixture in order to stabilize the mixture and optionally, where the reagent mixture is dried onto the device of the invention, to provide porosity in the dried mixture. Examples of suitable excipients include sugars such as mannitol, inositol and lactose, and PEG. Glycine can also be used as an excipient. Buffers may also be included to provide the required pH for optimal enzyme activity. For example, a Tris buffer (pH9) may be used. Stabilisers may be added to enhance, for example, enzyme stability. Examples of suitable stabilisers are amino acids, e.g. glycine, and ectoine.

Typically, the sample contacts all of the reagents in a single step. Therefore, a reagent mixture is provided which contains all of the required reagents and which can easily be contacted with the sample in order to carry out the assay. In a preferred embodiment, the reagent mixture for the HDL cholesterol assay of the invention comprises a surfactant which selectively breaks down high density lipoproteins; cholesterol esterase or a lipase; cholesterol dehydrogenase; NAD⁺ or an analogue thereof; a reductase; and a redox agent. In a more preferred embodiment, the reagent mixture for the HDL cholesterol assay comprises a surfactant which selectively breaks down high density lipoproteins, cholesterol esterase or a lipase, cholesterol dehydrogenase, NAD⁺ or an analogue thereof, diaphorase or putidaredoxin reductase and Ru(NH₃)₆³⁺.

The present invention also involves an assay for the total cholesterol content of the sample. Any method of electrochemically detecting total cholesterol content may be employed. In a preferred method, a surfactant which breaks down all lipoproteins is added to the sample, making the cholesterol and cholesterol esters bound to all lipoproteins available for reaction. The sample is then reacted with a cholesterol ester hydrolysing reagent and cholesterol oxidase or cholesterol dehydrogenase, and typically with a coenzyme and a redox agent and optionally a reductase. The measurement of cholesterol using cholesterol ester hydrolysing reagent, cholesterol oxidase or cholesterol dehydrogenase and optional other ingredients is typically carried out in the same manner as that described above for the HDL cholesterol test.

In one embodiment of the invention, the total cholesterol test is carried out using a separate series of reagents from the HDL cholesterol test. In this embodiment, sample is added to the first series of reagents to carry out the HDL cholesterol test and a separate portion of sample is added to a second series of reagents to carry out the total cholesterol test. The surfactants employed in the total cholesterol assay are typically bile acid derivatives or salts thereof, since these have been found to effectively break down all types of lipoproteins. Examples of suitable bile acid derivatives include cholic acid, taurocholic acid, glycocholic acid, lithocholic acid, deoxycholic acid, CHAPS (3-[(3-cholamidopropyl)-dimethylammonio]propane), CHAPSO, BIGCHAP and deoxy BIGCHAP. Combinations of two or more bile acid derivatives or their salts may also be used. For example, use of CHAPS alone has been found to occasionally cause precipitation of the enzymes present. Therefore, a combination of CHAPS with a different surfactant which does not have this effect, e.g. deoxy BIGCHAP, has been found to be beneficial. In a preferred embodiment, CHAPS and deoxy BIGCHAP are used in a 1:1 ratio. Glucopyranosides may also be used as the surfactant, e.g. n-nonyl-β-D-glucopyranoside.

The total amount of surfactant present is typically from 60 to 85%, preferably from 70 to 80% by weight relative to the weight of the cholesterol dehydrogenase enzyme

The reagents employed in the total cholesterol test are typically provided in the form of a single reagent mixture, which can be reacted with the sample in a single step. The reagent mixture preferably comprises a bile acid derivative or a salt thereof; cholesterol esterase or a lipase; cholesterol dehydrogenase; NAD⁺ or an analogue thereof; a reductase; and a redox agent. In a more preferred embodiment, the reagent mixture for the total cholesterol assay comprises CHAPS, deoxy BIGCHAP, cholesterol esterase, cholesterol dehydrogenase, TNAD⁺, putidaredoxin reductase and Ru(NH₃)₆³⁺.

In another embodiment of the invention, the same reagents are used to carry out the HDL cholesterol test and the total cholesterol test. In this embodiment, a selective reagent (e.g. a selective surfactant) as described above is used to provide a measure of the HDL cholesterol content of the sample. The selective reagent used, however, is one which does not entirely suppress reaction with other lipoproteins, but which preferentially reacts with HDL. In this way, the HDL cholesterol test is first carried out by measuring the electrochemical response of the sample/reagent mixture at a time when HDL preferentially reacts. Reaction is then continued and a second electrochemical measurement is taken at a time when all lipoproteins react. In this way a single reagent mixture in a single electrochemical cell may be used to provide a first measurement corresponding to the HDL content of the sample and a second measurement corresponding to the total cholesterol content of the sample.

The times at which the two measurements corresponding to HDL and total cholesterol contents may be taken will depend on the kinetics of the particular selective reagent used. The skilled person would be able to determine appropriate time points for measurement for any particular selective reagent. In many cases, the HDL cholesterol test will be completed within about a minute, e.g. within 30 seconds of addition of sample to the reagent mixture and the total cholesterol test will be completed after about 2 minutes from addition of sample to the reagent mixture.

The reagent mixtures for each of the HDL cholesterol assay and, if used, the total cholesterol assay are typically provided in solid form, for example in dried form, or as gel. Optionally, the reagents may be freeze dried. Alternatively they may be in the form of a solution or suspension. Whilst the amounts of each of the components present in the reagent mixtures are expressed above in terms of molarity or w/v, the skilled person would be able to adapt these amounts to suitable units for a dried mixture or gel, so that the relative amounts of each component present remains the same.

In a preferred embodiment of the invention, a portion of the sample is reacted with the reagents for the HDL cholesterol assay and simultaneously, or substantially simultaneously (e.g. within 30 seconds or within 15 seconds), a further portion of the sample is reacted with the reagents for the total cholesterol assay. This enables a calculation of the non-HDL cholesterol content of the sample to be determined in a very short period of time. In a preferred embodiment, the HDL cholesterol and total cholesterol assays are completed within 5 minutes, preferably within 3 minutes, 2 minutes, 90 seconds or 60 seconds from addition of the sample to the reagents.

Where an electrochemical measurement is carried out on whole blood, the measurement obtained may depend on the hematocrit. The measurement should therefore ideally be adjusted to at least partially account for this factor. Alternatively, the red blood cells can be removed by filtering the sample prior to carrying out the assay.

The kit of the present invention comprises a device having at least one, for example at least two, electrochemical cells, each cell having a working electrode, a reference or pseudo reference electrode and optionally a separate counter electrode. A series of reagents is associated with the or each cell in order that the cell(s) provide the desired electrochemical test. By the reagents being associated with the electrochemical cell, we mean that the reagents are positioned in such a way that once the sample contacts the reagents, the mixture of reagents and sample will contact the working electrode of the electrochemical cell. The kit also comprises a power supply for applying a potential across each cell and a measuring instrument for measuring the resulting electrochemical response of each cell.

The device of the invention has at least one electrochemical cell which is associated with the first series of reagents described above for carrying out the HDL cholesterol assay. In one embodiment, this single cell is used to carry out the HDL cholesterol test and the total cholesterol test. In an alternative embodiment, the device comprises at least a second electrochemical cell associated with the second series of reagents described above for carrying out the total cholesterol assay. The kit also comprises calculating means, typically a computer program, for determining the non-HDL cholesterol content of the sample by subtracting the HDL cholesterol result from the total cholesterol result.

Typically, each series of reagents is provided in the device in the form of a single reagent mixture (i.e. one reagent mixture is provided for each series of reagents). The reagent mixtures may be present in either liquid or solid form, but are preferably in solid form. Typically, the reagent mixtures are inserted into or placed onto the device whilst suspended/dissolved in a suitable liquid (e.g. water) and then dried in position. This step of drying the material into/onto the device helps to keep the material in the desired position, and helps to prevent reagent from migrating from one electrochemical cell of the device to another. Drying may be carried out, for example, by air-drying, vacuum drying, freeze drying or oven drying (heating). The reagent mixture is typically located in the vicinity of the electrodes, such that when the sample contacts the reagent mixture, contact with the electrodes also occurs.

The device may optionally comprise a membrane through which the sample to be tested passes prior to contact with the reagent mixtures. The membrane may, for example, be used to filter out components such as red blood cells, erythrocytes and/or lymphocytes. Suitable filtration membranes, including blood filtration membranes, are known in the art. Examples of blood filtration membranes are Presence 200 and PALL BTS SP300 of Pall filtration, Whatman VF2, Whatman Cyclopore, Spectral NX and Spectral X. Fibreglass filters, for example Whatman VF2, can separate plasma from whole blood and are suitable for use where a whole blood specimen is supplied to the device and the sample to be tested is plasma.

Alternative or additional membranes may also be used, including those which have undergone a hydrophilic or hydrophobic treatment prior to use. Other surface characteristics of the membrane may also be altered if desired. For example, treatments to modify the membrane's contact angle in water may be used in order to facilitate flow of the desired sample through the membrane. The membrane may comprise one, two or more layers of material, each of which may be the same or different. For example, conventional double layer membranes comprising two layers of different membrane materials may be used.

Appropriate devices for use in the present invention include those described in WO 2003/056319 and WO 2006/000828.

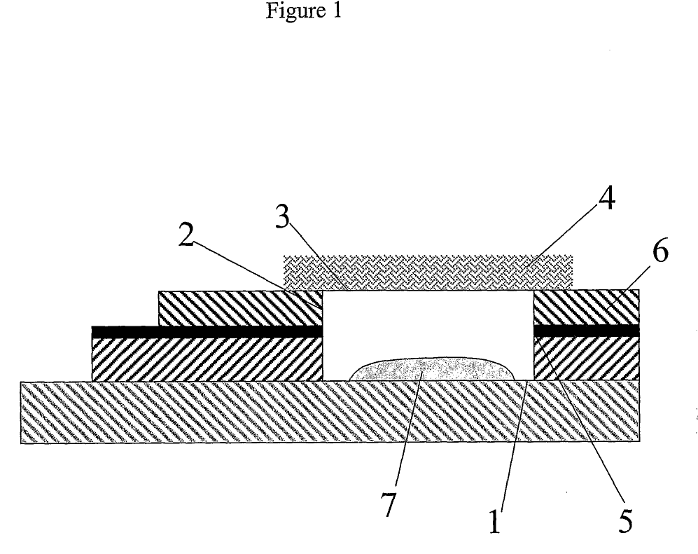

An electrochemical cell of a device according to one embodiment of the invention is depicted in FIG. 1. In this embodiment, the working electrode 5 is a microelectrode. For the purposes of this invention, a microelectrode is an electrode having at least one working dimension not exceeding 50 μm. The microelectrodes of the invention may have a dimension which is macro in size, i.e. which is greater than 50 μm.

The cell is in the form of a receptacle or a container having a base 1 and a wall or walls 2. Typically, the receptacle will have a depth (i.e. from top to base) of from 25 to 1000 μm. In one embodiment, the depth of the receptacle is from 50 to 500 μm, for example from 100 to 250 μm. In an alternative embodiment, the depth of the receptacle is from 50 to 1000 μm, preferably from 200 to 800 μm, for example from 300 to 600 μm. The length and width (i.e. from wall to wall), or in the case of a cylindrical receptacle the diameter, of the receptacle is typically from 0.1 to 5 mm, for example 0.5 to 1.5 mm, such as 1 mm.

The open end of the receptacle 3 may be partially covered by an impermeable material or covered by a semi-permeable or permeable material, such as a semi-permeable or permeable membrane. Preferably, the open end of the receptacle is substantially covered with a semi-permeable or permeable membrane 4. The membrane 4 serves, inter alia, to prevent dust or other contaminants from entering the receptacle.

The working electrode 5 is situated in a wall of the receptacle. The working electrode is, for example, in the form of a continuous band around the wall(s) of the receptacle. The thickness of the working electrode is typically from 0.01 to 25 μm, preferably from 0.05 to 15 μm, for example 0.1 to 20 μm. Thicker working electrodes are also envisaged, for example electrodes having a thickness of from 0.1 to 50 μm, preferably from 5 to 20 μm. The thickness of the working electrode is its dimension in a vertical direction when the receptacle is placed on its base. The thickness of the working electrode is its effective working dimension, i.e. it is a dimension of the electrode which contacts the sample to be tested. The working electrode is preferably formed from carbon, palladium, gold or platinum, for example in the form of a conductive ink. The conductive ink may be a modified ink containing additional materials, for example platinum and/or graphite. Two or more layers may be used to form the working electrode, the layers being formed of the same or different materials.

The cell also contains a pseudo reference electrode (not depicted) which may be present, for example, in the base of the receptacle, in a wall or walls of the receptacle or in an area of the device surrounding or close to the receptacle. The pseudo reference electrode is typically made from Ag/AgCl, although other materials may also be used. Suitable materials for use as the pseudo reference electrode will be known to the skilled person in the art. In this embodiment, the cell is a two-electrode system in which the pseudo reference electrode acts as both counter and reference electrodes. Alternative embodiments in which the cell comprises a reference electrode and a separate counter electrode can also be envisaged.

The pseudo reference (or reference) electrode typically has a surface area which is of a similar size to or smaller than, or which is larger than, for example substantially larger than, that of the working electrode 5. Typically, the ratio of the surface area of the pseudo reference (or reference) electrode to that of the working electrode is at least 1:1, for example at least 2:1 or at least 3:1. A preferred ratio is at least 4:1. The pseudo reference (or reference) electrode may, for example, be a macroelectrode. Preferred pseudo reference (or reference) electrodes have a dimension of 0.01 mm or greater, for example 0.1 mm or greater. This may be, for example, a diameter of 0.1 mm or greater. Typical areas of the pseudo reference (or reference) electrode are from 0.001 mm² to 100 mm², preferably from 0.1 mm² to 60 mm², for example from 1 mm² to 50 mm². The minimum distance between the working electrode and the pseudo reference (or reference) electrode is, for example from 10 to 1000 μm.

In order that the cell can operate, the electrodes must each be separated by an insulating material 6. The insulating material is typically a polymer, for example, an acrylate, polyurethane, PET, polyolefin, polyester or any other stable insulating material. Polycarbonate and other plastics and ceramics are also suitable insulating materials. The insulating layer may be formed by solvent evaporation from a polymer solution. Liquids which harden after application may also be used, for example varnishes. Alternatively, cross-linkable polymer solutions may be used which are, for example, cross-linked by exposure to heat or UV or by mixing together the active parts of a two-component cross-linkable system. Dielectric inks may also be used to form insulating layers where appropriate. In an alternative embodiment, an insulating layer is laminated, for example thermally laminated, to the device.

The electrodes of the electrochemical cell may be connected to any required measuring instruments by any suitable means. Typically, the electrodes will be connected to electrically conducting tracks which are, or can be, themselves connected to the required measuring instruments.

The required reagents are typically contained within the receptacle, as depicted at 7 in FIG. 1. Typically, the reagents, in the form of a single reagent mixture, are inserted into the receptacle in liquid form and subsequently dried to help immobilise the composition. The reagent mixture, for example, may be air-dried, vacuum dried, freeze dried or oven-dried (heated), most preferably it is freeze dried.

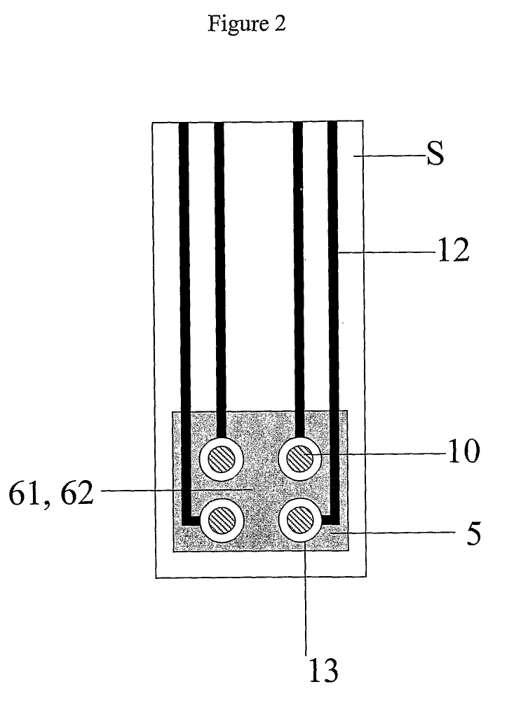

The device of the invention comprises one or more electrochemical cells. A device, having four electrochemical cells 10 on a strip S, is depicted in FIG. 2. Each cell comprises a working electrode and may additionally comprise a counter electrode. Preferably, and as depicted in FIG. 2, a single layer of pseudo reference electrode material 5 is provided on the surface of the strip 61,62, surrounding each receptacle and leaving a blank area 13 between the perimeter of the receptacle and the edge of the pseudo reference electrode layer. The electrodes are connected to the required instruments via conductive tracks 12.

This embodiment of the invention allows a number of measurements to be taken simultaneously. In a preferred aspect of this embodiment, one of the cells contains a reagent mixture for carrying out the HDL cholesterol test and a second cell contains reagents for carrying out the total cholesterol test.

A further cell may be used as a control cell. The control cell typically comprises a further series of reagents comprising a surfactant, coenzyme, redox agent and optionally a reductase as well as buffers, stabilisers and excipients as desired. Typically, the control reagents are the same reagents, or very similar reagents, to those used to carry out the HDL cholesterol and total cholesterol tests, with the exception that the enzymes reactive with the cholesterol are not present. Reaction of the sample with the control reagents, and subsequent measurement of any electrochemical response, enables the skilled person to determine the response due to interfering substances in the sample. The response due to interferents can subsequently be subtracted from the measurements of the total cholesterol and HDL cholesterol tests to give more accurate results wherein the effects of interferents are reduced or eliminated. Further, should the sample tested contain any significant quantities of interfering substances which will cause the test to fail, this can be identified using the control reaction.

The kit of the invention may comprise a strip S containing the electrochemical cell(s) (e.g. that depicted in FIG. 2 and described above) and an electronics unit, e.g. a hand-held portable electronics unit, capable of forming electronic contact with the strip S. The electronics unit may, for example, house the power supply for providing a potential to the electrodes, as well as a measuring instrument for detecting an electrochemical response and any other measuring instruments required. The electronics unit may also include a calculator for determining the non-HDL cholesterol content. One or more of these systems may be operated by a computer program.

The device of the present invention is operated by providing a sample to the device and enabling the sample to contact each reagent mixture. In the case of reagent mixtures which are in solid form, a wet-up time of approximately 20 seconds is typically provided to enable the reagent mixtures to be dissolved/suspended in the sample and to allow reaction to occur. This wet-up time may be varied, however, depending on the nature of the device used. For example, where a membrane is present to provide filtration of a sample prior to contact with the reagents, a wet-up time of up to 5 minutes may be used. The sample/reagent mixtures should be in electronic contact with the corresponding working electrode in order that electrochemical reaction can occur at the electrode.

A potential is then applied across each cell and the electrochemical response of each cell is measured. This is typically achieved by measurement of the current. Typically, the potential is applied and measurement made after a period of from 10 seconds to 500 seconds, for example from 10 seconds to 400 seconds or 10 seconds to 180 seconds from the time at which the sample and reagents are mixed, e.g. a period of at least 10 or 15 seconds, and of up to 90, 60 or 30 seconds, for example approximately 20 seconds. The use of periods within this preferred range helps to ensure that the HDL cholesterol assay detects only cholesterol bound to HDL.

Where a single cell is used to measure the HDL cholesterol and total cholesterol contents of the sample, a second potential is applied across the cell after a further time lapse. For example the second potential may be applied at least 120 seconds or at least 150 or 180 seconds from the time at which the sample and reagents are mixed. Measurement of the current during this second applied potential provides a result corresponding to the total cholesterol content.

Typically, where Ru(II) is the product to be detected at the working electrode, the potential applied to the cell is from 0.1V to 0.3V. A preferred applied potential is 0.15V. (All voltages mentioned herein are quoted against a Ag/AgCl reference electrode with 0.1M chloride). In a preferred embodiment, the potential is stepped first to a positive applied potential of 0.15V for a period of about 1 second. A negative potential of −0.4 to −0.6V is then applied when it is desired to measure the reduction current. The use of the double potential step is described in WO 03/097860 incorporated herein by reference. Where a different redox agent is used, the applied potentials can be varied in accordance with the potentials at which the oxidation/reduction peak occurs.

The electrochemical test of the invention therefore enables a measurement of non-HDL cholesterol to be made in a very short period of time, typically within about 5 minutes, preferably within 3 minutes, 2 minutes or even 1 minute from application of a sample to the device. Results may in some circumstances be available in as little as 15 or 30 seconds from application of a sample to the device.

### EXAMPLES

### Example 1

### Analysis of HDL-Selective Surfactants

In this Example, a testing protocol is described to determine whether a surfactant selectively breaks down HDL cholesterol and is therefore appropriate for use in the HDL cholesterol assay of the invention.

### Preparation of Buffer Solution (Tris Buffer-5% Glycine pH9.0)

Trizma Pre-Set Crystals containing crystallised Tris and Tris HCl (pH 9.0, Sigma, T-1444) were dissolved in 950 mls dH₂O and the pH recorded. Following this 50 g of Glycine (Sigma, G-7403) was added to the tris solution and the pH recorded. The pH was then adjusted to within 8.8-9.2 using 10M Potassium hydroxide (Sigma, P-5958) and the solution made up to 1000 mls with dH₂O and the final pH recorded (pH9.1). The solution was stored at 4° C. with an expiry date of 1 month.

### Preparation of Surfactant Solution

A surfactant solution was prepared by addition of Amphitol 20N (Kao) to the pre-prepared buffer solution to yield a 10% Amphitol 20N buffered solution.

### Preparation of LDL & HDL Samples

LDL (Scipac, P232-8) and HDL (Scipac, P233-8) samples were made at 10× the required concentration (due to a 1:10 dilution in the final testing mixture) using dilipidated serum (Scipac, S139). The samples were then analysed using a Space clinical analyser (Schiappanelli Biosystems Inc). The approximate concentrations of LDL and HDL were 10 mM, giving 1 mM in the final testing mix.

### Preparation of Enzyme Mixture

An enzyme mixture was prepared by adding the following to the pre-prepared buffer solution:

### 160 mM Ruthenium Hexaammine (III) Chloride (Alfa Aesar, 10511)

17.7 mM Thionicotinamide adenine dinucleotide (Oriental Yeast Co)
8.4 mg/ml Putidaredoxin Reductase (Biocatalysts)
6.7 mg/ml Cholesterol Esterase (Sorachim/Toyobo, COE-311)
44.4 mg/ml Cholesterol Dehydrogenase, Gelatin free (Amano, CHDH-6)

### Testing Protocol

9 μl of the surfactant solution was mixed with 9 μl of the enzyme mixture. At T=−30 seconds, 2 μl of sample (either 10× concentrated LDL or 10× concentrated HDL, or 2 μl of delipidated serum) was mixed with the resulting surfactant:enzyme mix and 9 μl of the resulting solution placed onto an electrode. At T=0 seconds the chronoamperometry test was initiated. An oxidation potential of 0.15V was applied followed by a reduction potential of −0.45V. During application of the oxidation potential, the current was measured at 5 time points (T=0, 32, 63, 90 and 118 seconds). Each sample was tested in duplicate.

### Results

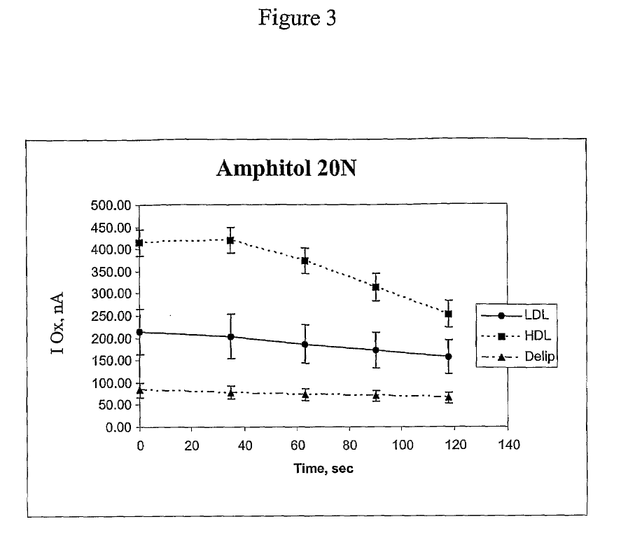

The results of the HDL and LDL test are depicted in FIG. 3, together with the delipidated sample as control. It is clear from FIG. 3 that Amphitol 20N is selective for HDL over LDL, especially at short time periods.

In order to determine the differentiation value, the measured HDL current was plotted against known HDL concentration and the gradient at each time point was measured. Similarly, the gradient for each LDL measurement was calculated. At T=32 seconds (in total 62 seconds after addition of the sample to the reagents), the LDL gradient (G_{HDL}) was 433.08 based on the known HDL concentration of 0.796 mM, the LDL gradient (G_{LDL}) was 171.10 based on the known LDL concentration of 0.744 mM and the resulting differentiation value was 60.49.

Some differentiation values for other selective surfactants are given in Table 1 below, all measured at T=32 seconds:

TABLE 1

| | Differentiation at |
|---|---|
| Surfactant | T = 32 seconds |
| Amphitol 20N | 60.49 |
| Brij | 32.14 |
| Chemal LA9 | 56.04 |
| Emulgen 109P | 59.29 |
| Emulgen 1135S-70 | 95.99 |
| Emulgen A-60 | 67.70 |
| Emulgen A-90 | 57.14 |
| HEGA-8 | 84.49 |
| HEGA-9 | 80.19 |
| C-HEGA-9 | 74.98 |
| Newkalgen FS12 | 66.00 |
| Silwet L7600 | 60.31 |
| Softazoline LPBR | 33.73 |
| Surfynol 485 STK | 53.92 |
| sucrose | 73.65 |
| monocaprate | |
| Surfactant 10G | 50.52 |
| Zonyl FSN100 | 46.13 |

### Example 2

### Determination of Non-HDL Cholesterol Content

Electrochemical sensor strips in accordance with FIG. 2 were used. Each strip contained two electrochemical cells in accordance with FIG. 1.

The first electrochemical cell contained a formulation for carrying out a total cholesterol test. The formulation was as follows:

0.1M tris (pH 9.0), 50 mM MgSO₄, 5% w/v glycine, 1% w/v myo-inositol, 1% w/v ectoine, 5% w/v CHAPS, 5% w/v deoxy bigCHAP, 80 mM Ru(NH₃)₆Cl₃, 8.8 mM TNAD, 4.4 mg/mL PdR, 3.3 mg/mL ChE and 66 mg/mL gelatin free ChDH.

The second electrochemical cell contained a formulation for carrying out an HDL cholesterol test. The formulation was as follows:

0.1 M tris (pH 9.0), 2% W/V MgCl₂, 3% w/v glycine, 1% w/v hydroxy ectoine, 1% w/v lactitol, 1% w/v lactose, 2% w/v Emulgen B66, 20 mM Ru(NH₃)₆Cl₃, 1.66 mM TNAD, 2 mg/mL PdR, 3.3 mg/mL Genzyme lipase from Chromobacterium viscosum, 20 mg/mL gelatin free ChDH.

Each formulation was made up as an aqueous solution/suspension, inserted into the respective electrochemical cell and dried.

Experiments were performed over 2 days, with 10 donors on day 1 and 20 donors on day 2. Donors were not fasted. Whole blood samples were collected into Li heparin vacutainer tubes. These samples were centrifuged and the plasma collected.

The total cholesterol and HDL cholesterol contents of each sample were tested using the pre-prepared electrochemical sensor strips. 20 uL plasma was applied to each strip in order to fill each electrochemical cell. An oxidation potential of 0.15V followed by a reduction potential of −0.45V was applied at the following times after sample application: HDL: 36s; Total Cholesterol: 110s. Both oxidation and reduction current valves were measured. 8 repeat measurements with individual sensor strips were performed with each sample.

The concentrations of total cholesterol, HDL cholesterol and LDL cholesterol in each sample were also determined using a Randox SPACE clinical analyzer. Each measurement on the analyzer was made in duplicate and the average concentration value determined.

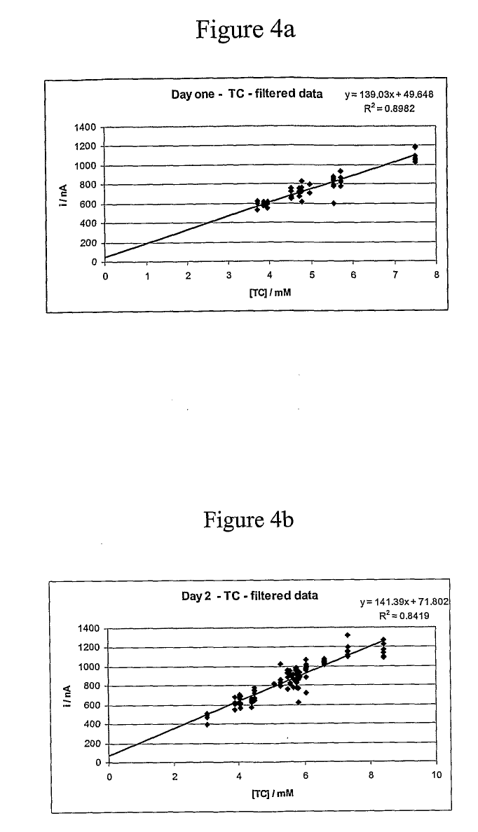

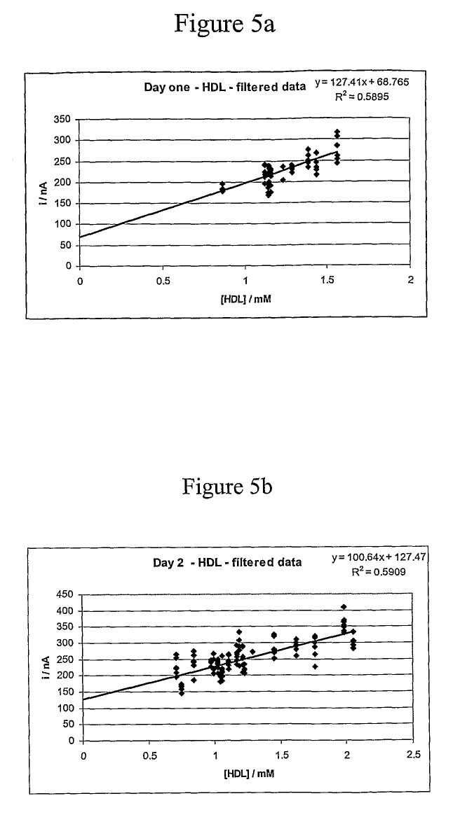

The results of the total cholesterol (TC) tests are depicted in FIGS. 4a and 4b (days one and two respectively). These Figures show the oxidation current responses plotted against the TC content determined by the Randox SPACE clinical analyzer. Similarly, the results of the HDL cholesterol tests are depicted in FIGS. 5a and 5b, with the oxidation current plotted against the HDL cholesterol content determined by the Randox SPACE clinical analyzer.

Linear regression was performed for each graph. The slope and intercept from the best line fit of each graph was used to predict the analyte concentration for each current response:

[analyte]=(i_{analyte}−intercept)/slope

The calculated analyte values for TC and HDL cholesterol were used to calculate the concentration of non-HDL cholesterol according to the equation below:

[Non-HDL]=[TC]−[HDL].

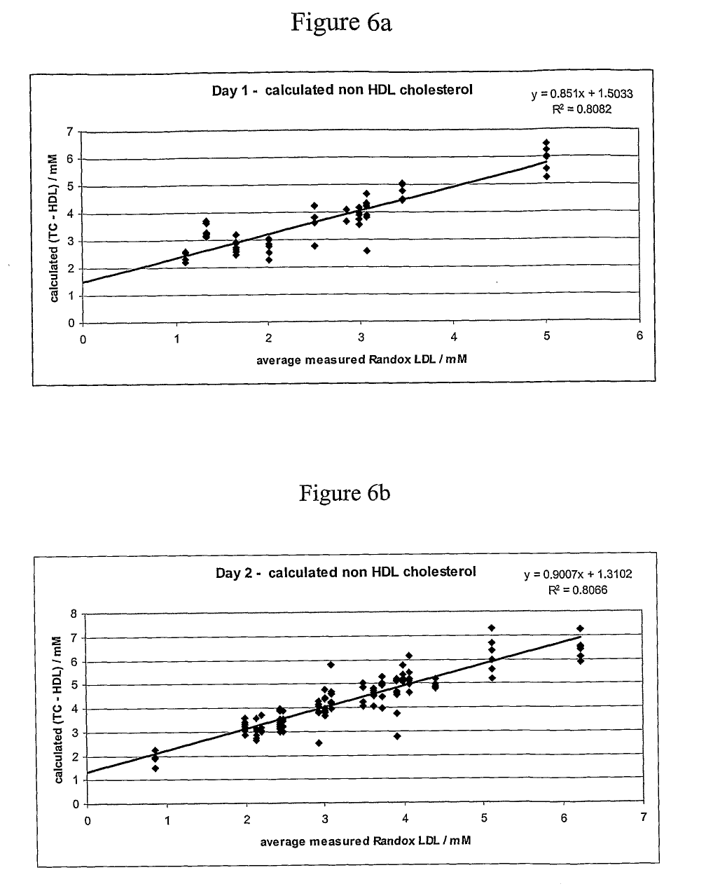

Plots of [non-HDL] (y-axis) as determined from the measured HDL cholesterol and TC of each sample vs. [LDL] (x-axis) as determined by the Randox SPACE clinical analyzer are shown in FIGS. 6a and 6b. Good correlation between non-HDL cholesterol concentration and LDL cholesterol concentration was observed.

The non-HDL cholesterol content as measured in accordance with the present invention was also compared to the LDL cholesterol content as determined using the Friedwald equation:

$$[ LDL ] = [ Totalcholesterol ] - [ HDL ] - \frac{[ Triglyceride ]}{2.17}$$

wherein [Totalcholesterol] is the total concentration of cholesterol in the sample, [HDL] is the concentration of cholesterol bound to high density lipoproteins and [Triglyceride] is the triglyceride concentration of the sample, the concentrations being measured in mmol dm⁻³.

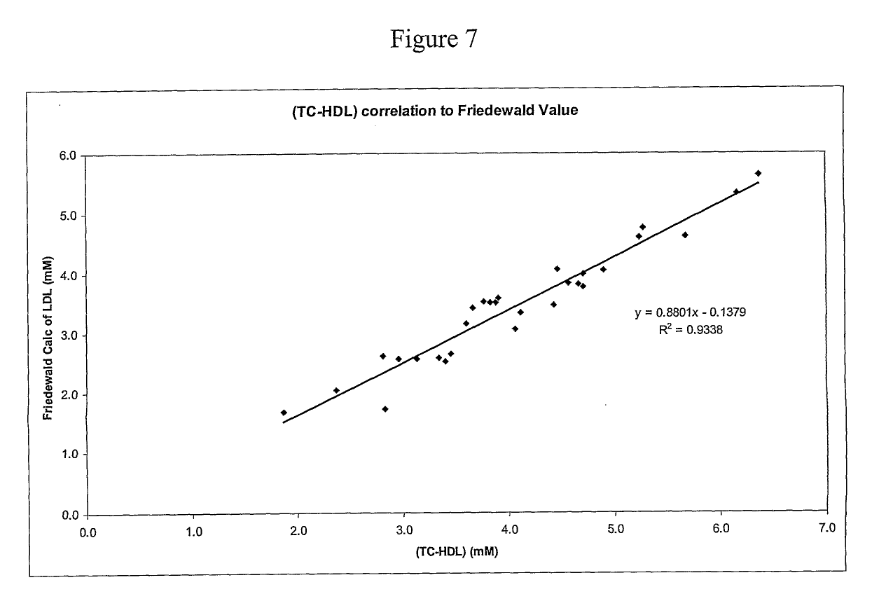

The results are shown in FIG. 7 plotted against the non-HDL cholesterol content. A good correlation can be seen between non-HDL cholesterol content and LDL cholesterol content determined using the Friedwald equation.

### Example 3

### MEGA-8

The aim of the experiment was to investigate the use of a single sensor containing 100 mM MEGA-8 for determining the response to LDL, by using the response to HDL at short measurement times and to TC at long measurement times.

### 10% β-Lactose Buffer;

A buffer solution was prepared containing 0.1M Tris pH9.0, 30 mM KOH and 10% β-lactose

### 30 mM Ru Acac Solution:

30 mM RuAcac solution was made up by addition of RuAcac to the above 10% lactose buffer. This solution was mixed using a Covaris acoustic mixer.

### Ru Acac=Cis-[Ru(acac)₂(Py-3-CO₂H)(Py-3-CO₂)]

### MEGA Solutions

Double strength MEGA solution was made by addition of MEGA to the above RuAcac solution to produce the following final concentrations:

### MEGA-8 (Soltec Ventures S116)

200 mM (0.0188 g in 292 μl RuAcac solution)

### Enzyme Mixture

Enzyme mixture was made at double strength by addition of enzyme to the RuAcac solution to produce the following final concentrations:

17.7 mM Thionicotinamide adenine dinucleotide (Oriental Yeast Co)
8.4 mg/ml Putidaredoxin Reductase (Biocatalyst)
6.7 mg/ml Lipase (Genzyme)
44.4 mg/ml Cholesterol Dehydrogenase, Gelatin free (Amano, CHDH-6)
This solution was mixed using a Covaris acoustic mixer.

### Dispense and Freeze Drying

For the final enzyme solution, equal volumes (approximately 50 ul) of double concentration enzyme solution and MEGA solution were mixed 1:1 to give the final enzyme/surfactant mix. 0.4 μl/well of each solution was dispensed onto sensors as described in WO 0356319 using an electronic pipette. The dispensed sensor sheets were then freeze dried.

### Plasma Samples

Plasma samples were defrosted for 30 minutes before being centrifuged for 5 minutes at 2900 RCF. Delipidated serum (Scipac, S139) was also used as a sample. The samples were analysed using a Space clinical analyser (Schiappanelli Biosystems Inc) for TC, TG, HDL and LDL concentrations.

### Testing Protocol

12-15 μl of a plasma sample was used per electrode. On the addition of plasma the chronoamperometry test was initiated using a multiplexer attached to an Autolab. The oxidation current is measured at 0.15 V at 13 time points (0, 32, 64, 96, 128, 160, 192, 224, 256, 288, 320, 352 and 384 seconds), with a reduction current measured at −0.45 V at the final time point (416 seconds). Each sample was tested in duplicate.

### Analysis

Calibration plots were constructed to HDL or TC at each time point.

### Results

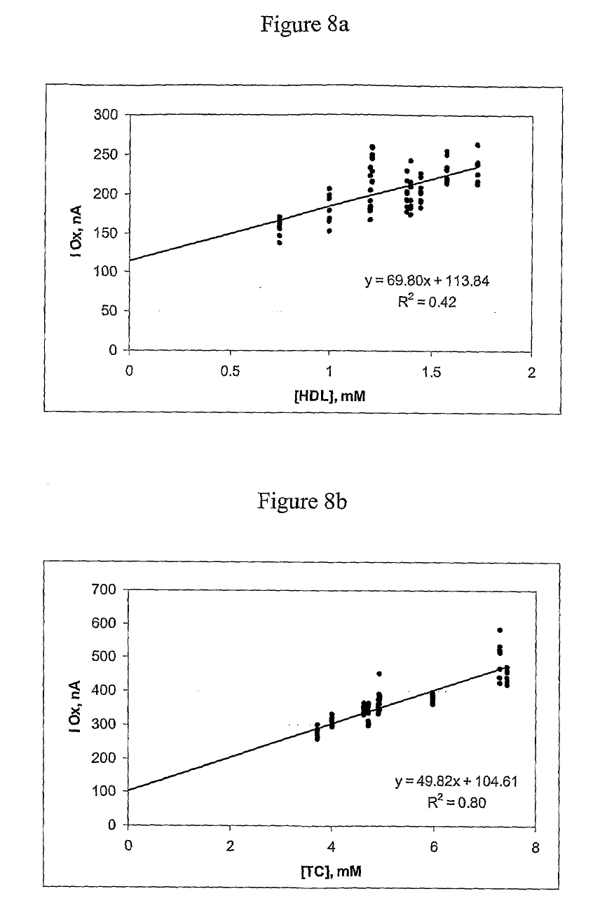

Good calibration plots to HDL at 32 sec and to TC at 384 sec were obtained. These are shown in FIG. 8.

For each individual electrode, the current value at 32 sec was used to calculate the HDL value predicted by the HDL calibration plot, using the gradient and intercept values. Similarly, for each individual electrode, the current value at 384 sec was used to calculate the TC value predicted by the TC calibration plot, using the gradient and intercept values.

For each individual electrode, these predicted values of HDL and TC were used to calculate values of TC-HDL. These values of TC-HDL were plotted against the LDL value of the plasma sample as measured on the Randox analyser.

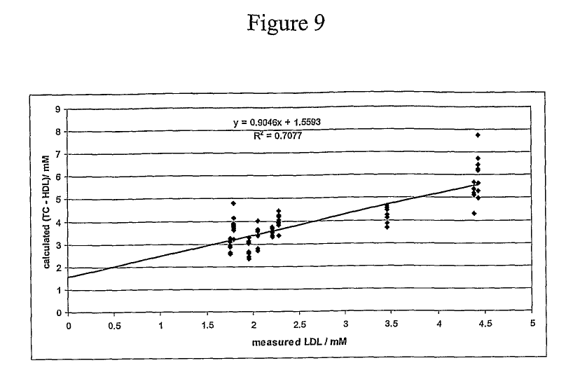

The plot of calculated TC-HDL vs. Randox LDL is shown in FIG. 9. The calculated values of TC-HDL give reasonable correlation with the values of LDL measured by the reference method.

### Conclusions

A single sensor based on 100 mM MEGA-8 can be used to calculate the response to LDL, by measuring the response to HDL at short times, and TC at long times, and calculating LDL=TC−HDL.

### Example 4

### Various Enzymes

The aim of the experiment was to investigate the determination of plasma LDL from TC-HDL values, using TC and HDL sensors prepared with different cholesterol ester hydrolyzing enzymes (lipase or cholesterol esterase) or different NADH oxidases (Putitadoxin reductase or diaphorase).

3 separate TC and 3 separate HDL sensors were prepared. The differences in the reagents used in each sensor are summarized in the table below:

| Sensor | Chemistry |
|---|---|
| TC 1 | 80 mM Ru(NH₃)₆Cl₃, 5% CHAPS, 5% deoxy bigCHAP, |
| | Toyobo cholesterol esterase and PdR |
| TC 2 | 80 mM Ru(NH₃)₆Cl₃, 5% CHAPS, 5% deoxy bigCHAP, |
| | Toyobo cholesterol esterase and diaphorase |
| TC 3 | 80 mM Ru(NH₃)₆Cl₃, 5% CHAPS, 5% deoxy bigCHAP, |
| | Toyobo lipase and PdR |
| HDL 1 | 30 mM RuAcac, 100 mM SMC, Genzyme lipase and PdR |
| HDL 2 | 30 mM RuAcac, 100 mM SMC, Genzyme lipase and |
| | diaphorase |
| HDL 3 | 30 mM RuAcac, 100 mM HEGA-9, Genzyme cholesterol |
| | esterase and PdR |

Two separate enzyme mixes were prepared as follows, one with 80 mM Ru(NH₃)₆Cl₃ mediator and one with 30 mM RuAcac mediator.

Enzyme mix with 80 mM Ru(NH₃)₆Cl₃ mediator:

### 10% β-Lactose Buffer;

A buffer solution was prepared containing 0.1M Tris pH9.0, 30 mM KOH and 10% β-lactose.

### Surfactant Solution

Double strength surfactant solution was made by addition of surfactant to the lactose solution to produce the following final concentrations.

### 10% CHAPS (Sigma-Aldrich, C5070)

0.05 g in 500 ul lactose solution.
10% deoxy bigCHAP (Soltec Ventures, S115)
0.038 g in 380 ul CHAPS solution.

### 80 mM Ru(NH₃ Cl₃ Solution:

80 mM Ru(NH₃)₆Cl₃ solution was made up by addition of Ru(NH₃)₆Cl₃ to the 10% CHAPS/DeoxyBIGCHAP solution.

### Enzyme Mixture

Enzyme mixture was made at double strength by addition of enzymes to the Ru(NH₃)₆Cl₃ solution to produce the following final concentrations:

17.7 mM Thionicotinamide adenine dinucleotide (Oriental Yeast Co)
8.4 mg/ml Putidaredoxin Reductase (Biocatalyst) or 8.4 mg/mL diaphorase (Unitika)
6.7 mg/ml Cholesterol esterase (Toyobo, COE-311) or lipase (Toyobo, LPL311)
44.4 mg/ml Cholesterol Dehydrogenase, Gelatin free (Amano, CHDH-6)

For each final enzyme solution, equal volumes (approximately 50 uL) of double concentration enzyme solution and surfactant solution were mixed 1:1 to give the final enzyme/surfactant mix.

Enzyme mix with 30 mM RuAcac mediator:

### 10% β-Lactose Buffer;

A buffer solution was prepared containing 0.1M Tris pH9.0, 30 mM KOH and 10% β-lactose

### 30 mM Ru Acac Solution:

30 mM RuAcac solution was made up by addition of RuAcac to the above 10% lactose buffer. This solution was mixed using a Covaris acoustic mixer.

### Ru Acac=Cis-[Ru(acac)₂(Py-3-CO₂H)(Py-3-CO₂)]

### Surfactant Solutions

Double strength surfactant solutions were made by addition of surfactant to the RuAcac solution to produce the following final concentrations:

### 200 mM SMC (Dojindo, SO21-12)

0.0298 g in 303 ul of RuAcac solution.

### 200 mM HEGA-9 (Anatrace, H109)

0.0149 g in 204 ul of RuAcac solution.

### Enzyme Mixture

Enzyme mixture was made at double strength by addition of enzymes to the RuAcac solution to produce the following final concentrations:

17.7 mM Thionicotinamide adenine dinucleotide (Oriental Yeast Co)
8.4 mg/ml Putidaredoxin Reductase (Biocatalyst) or diaphorase (Unitika)
20.1 mg/ml Lipase (Genzyme) or Cholesterol esterase (Genzyme)
44.4 mg/ml Cholesterol Dehydrogenase, Gelatin free (Amano, CHDH-6)
This solution was mixed using a Covaris acoustic mixer.

The dispense, freeze drying, preparation of plasma samples and testing protocol were as described in Example 3, except for sensors with HEGA-9, which sensors were tested with 6 repeat oxidations (at time points 0, 59, 118, 177, 236 and 295 seconds), with a reduction current measured at −0.45 V at the final time point (354 seconds). The transient time was 8 seconds.

### Analysis

Calibration plots were constructed to HDL or TC at each time point.

### Results

The responses of the TC sensors were plotted for TC1, TC2 and TC3 vs. TC concentration, at measurement times of 192, 224 or 224 seconds respectively. The responses of the HDL sensors were plotted vs. plasma HDL concentration, at measurement times of 384, 384 and 295 seconds for HDL1, HDL2, HDL3 respectively.

For each of these calibration plots, the slope and intercept of the best line fit was used to calculate the TC (or HDL) concentration of each current value. The concentration values were then averaged for each sample (n=8).

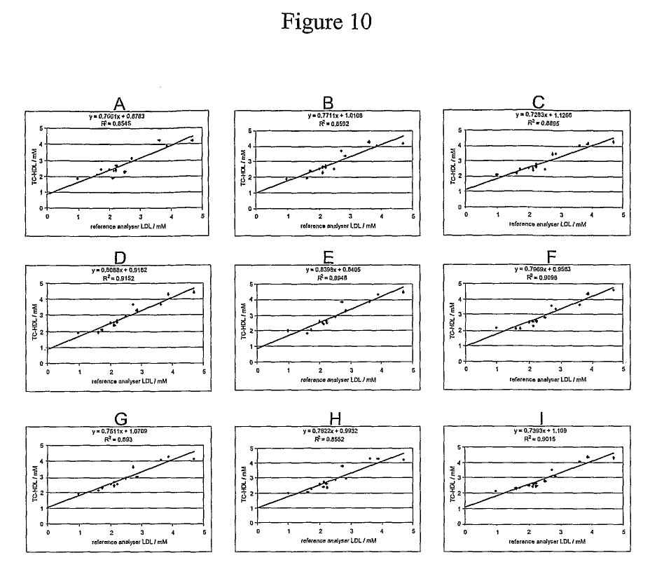

For all possible combinations of TC and HDL sensors, TC-HDL concentration values were calculated for each plasma sample and plotted against the LDL concentration of the sample measured by the lab analyser. These calibration plots are shown in FIG. 10.

FIG. 10 legend:

A: TC sensor 1 and HDL sensor 1.
B: TC sensor 1 and HDL sensor 2.
C: TC sensor 1 and HDL sensor 3.
D: TC sensor 2 and HDL sensor 1.
E: TC sensor 2 and HDL sensor 2.
F: TC sensor 2 and HDL sensor 3.
G: TC sensor 3 and HDL sensor 1.
H: TC sensor 3 and HDL sensor 2.
I: TC sensor 3 and HDL sensor 3.

### Conclusions

Good correlation is obtained between the calculated values of plasma TC-HDL and plasma LDL determined by the standard method, for TC and HDL sensors prepared with different enzymes.

### Example 5

### Various Surfactants

The aim of the experiment was to investigate the determination of plasma LDL from TC-HDL values, using TC and HDL sensors prepared with a range of surfactant types.

Two separate enzyme mixes were prepared, one with 80 mM Ru(NH₃)₆Cl₃ mediator and one with 30 mM RuAcac mediator. 80 mM Ru(NH₃)₆Cl₃ mediator and 30 mM RuAcac mediator solutions were prepared as described in Example 4.

Enzyme mix with 80 mM Ru(NH₃)₆Cl₃ mediator:

### Surfactant Solutions

Double strength surfactant solutions were made by addition of surfactant to the Ru(NH₃)₆Cl₃ solution to produce the following final concentrations:

### 10% CHAPSO (Sigma-Aldrich, C4695)

0.018 g in 180 ul Ru(NH₃)₆Cl₃ solution.
10% deoxy bigCHAP (Soltec Ventures, S115)
0.0227 g in 227 ul Ru(NH₃)₆Cl₃ solution.

### Enzyme Mixture

Enzyme mixture was made at double strength by addition of enzymes to the Ru(NH₃)₆Cl₃ solution to produce the following final concentrations:

17.7 mM Thionicotinamide adenine dinucleotide (Oriental Yeast Co)
8.4 mg/ml Putidaredoxin Reductase (Biocatalyst)
6.7 mg/ml Cholesterol esterase (Toyobo, COE-311)
44.4 mg/ml Cholesterol Dehydrogenase, Gelatin free (Amano, CHDH-6)

Enzyme mix with 30 mM RuAcac mediator:

### Surfactant Solutions

Double strength surfactant solutions were made by addition of surfactant to the RuAcac solution to produce the following final concentrations:

10% n-nonyl-β-D-glucopyranoside (Anatrace, N324)
0.0192 g in 192 ul of RuAcac solution.
100 mM sucrose monododecanoate (Calbiochem, 324374)
0.0091 g in 173 ul of RuAcac solution.

### 200 mM HEGA-9 (Anatrace, H109)

0.0148 g in 203 ul of RuAcac solution.
200 mM n-octyl-β-D-maltopyranoside (Anatrace, 0310)
0.0191 g in 211 ul RuAcac solution.

### Enzyme Mixture

Enzyme mixture was made at double strength by addition of enzymes to the RuAcac solution to produce the following final concentrations:

17.7 mM Thionicotinamide adenine dinucleotide (Oriental Yeast Co)
8.4 mg/ml Putidaredoxin Reductase (Biocatalyst)
20.1 mg/ml Lipase (Genzyme)
44.4 mg/ml Cholesterol Dehydrogenase, Gelatin free (Amano, CHDH-6)
This solution was mixed using a Covaris acoustic mixer.

The dispense, freeze drying, preparation of plasma samples and testing protocol were as described in Example 3 with a transient time of 4 seconds, except for sensors with HEGA-9 which were tested with 6 repeat oxidations (at time points 0, 59, 118, 177,236 and 295 seconds), with a reduction current measured at −0.45 V at the final time point (354 seconds). The transient time was 8 seconds. Each sample was tested in duplicate.

### Analysis

Calibration plots were constructed to HDL or TC at each time point.

### Results

The responses of sensors containing CHAPSO, deoxy bigCHAP or n-nonyl-β-D-glucopyranoside were plotted vs. TC concentration, at measurement times of 224, 160 and 288 seconds respectively. The responses of sensors containing sucrose dodecanoate, HEGA-9 or n-octyl-β-D-maltopyranoside were plotted vs. plasma HDL concentration, at measurement times of 384, 118 and 384 seconds respectively.

For each of these calibration plots, the slope and intercept of the best line fit was used to calculate the TC (or HDL) concentration of each current value. The concentration values were then averaged for each sample (n=8).

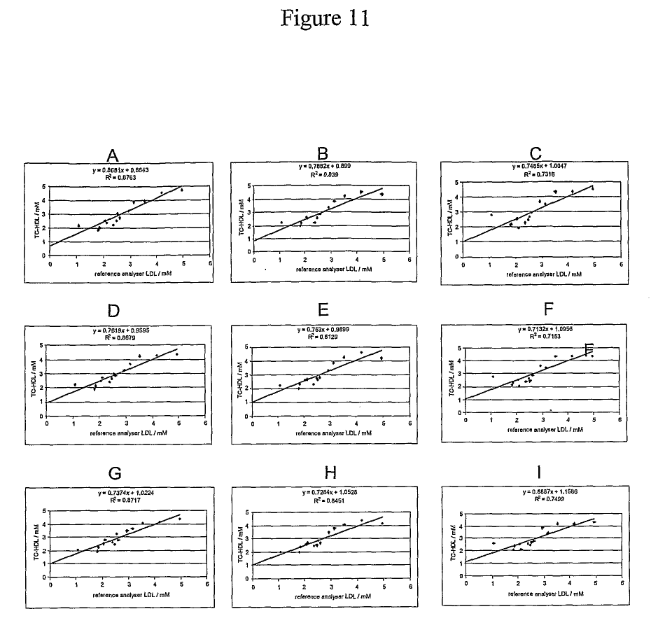

For all possible combinations of TC and HDL sensors, TC-HDL concentration values were calculated for each plasma sample and plotted against the LDL concentration of the sample measured by the lab analyser. These calibration plots are shown in FIG. 11.

FIG. 11 legend:

A: TC sensor contains 5% CHAPSO, HDL sensor contains 50 mM sucrose monododecanoate.
B: TC sensor contains 5% CHAPSO, HDL sensor contains 100 mM HEGA-9.
C: TC sensor contains 5% CHAPSO, HDL sensor contains 100 mM n-octyl-β-D-maltoside.
D: TC sensor contains 5% deoxy bigCHAP, HDL sensor contains 50 mM sucrose monododecanoate.
E: TC sensor contains 5% deoxy bigCHAP, HDL sensor contains 100 mM HEGA-9.
F: TC sensor contains 5% deoxy bigCHAP, HDL sensor contains 100 mM n-octyl-β-D-maltoside.
G: TC sensor contains 5% n-nonyl-β-D-glucopyranoside, HDL sensor contains 50 mM sucrose monododecanoate.
H: TC sensor contains 5% n-nonyl-β-D-glucopyranoside, HDL sensor contains 100 mM HEGA-9.
I: TC sensor contains 5% n-nonyl-β-D-glucopyranoside, HDL sensor contains 100 mM n-octyl-β-D-maltoside.

### Conclusions

Good correlation is obtained between the calculated values of plasma TC-HDL and plasma LDL determined by the standard method, for TC and HDL sensors prepared with different surfactants.

### Example 6

### Measurement of Non-HDL Cholesterol with Total Cholesterol and HDL Cholesterol Electrochemical Sensor Strips

Multianalyte electrochemical sensor strips for TC and HDL were prepared in house. Each sensor strip contained individual sensors for TC and HDL, comprising dried enzyme reagent and a screen printed microelectrode (for description on the sensor see WO200356319). Each sensor strip was individually packaged with desiccant.

The formulation used for the TC sensor was as follows:

0.1M tris (pH 9.0), 50 mM MgSO₄, 5% w/v glycine, 1% w/v myo-inositol, 1% w/v ectoine, 5% w/v CHAPS, 5% w/v deoxy bigCHAP, 80 mM Ru(NH₃)₆Cl₃, 8.8 mM TNAD, 4.2 mg/mL PdR, 3.3 mg/mL ChE and 66 mg/mL gelatin free ChDH.

The formulation used for the HDL sensor was as follows:

0.1 M tris (pH 9.0), 10% w/v lactose, 5% w/v sucrose monocaprate, 80 mM Ru(NH₃)₆Cl₃, 8.8 mM TNAD, 4.2 mg/mL PdR, 3.4 mg/mL Genzyme lipase from Chromobacterium viscosum, 22 mg/mL gelatin free ChDH.

Experiments were performed on 1 day, with 30 donors (10 fasting and 20 non-fasting). Whole blood samples were collected into Li heparin vacutainer tubes. These samples were centrifuged and the plasma collected. The concentrations of TC, HDL and LDL in each sample were determined using a Randox SPACE clinical analyzer. Each measurement on the analyzer was made in duplicate and the average concentration value used.

The electrochemical sensor responses were determined using 20 uL plasma per strip using an autolab and multiplexer. The oxidation current (nA) was measured at 15 time points, at 14 second intervals. 8 repeat measurements with individual sensors were performed with each sample.

Each sensor response was recorded as an oxidation current value (nA) at each time point.

### Results

Calibration graphs for TC and HDL were made of filtered oxidation current responses vs. concentration of analyte, using concentration values obtained from the Randox SPACE clinical analyser.

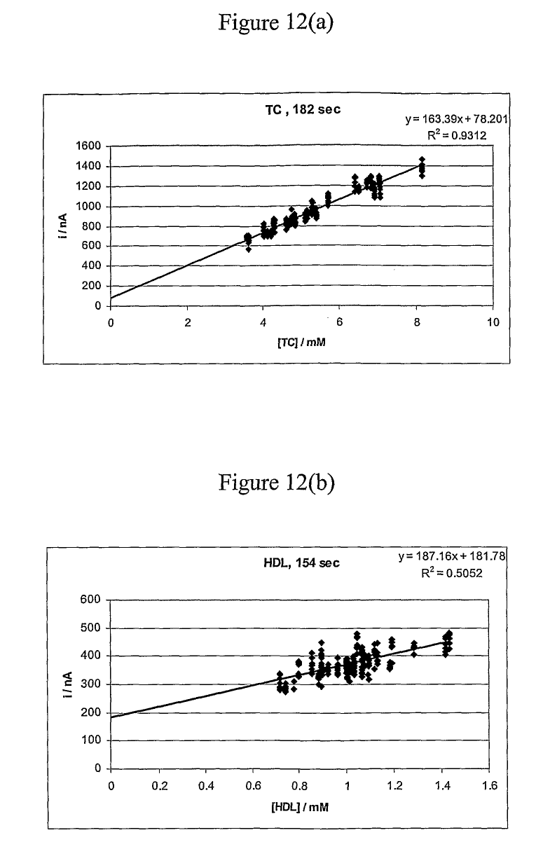

Linear regression was performed for each graph. The graphs are shown in FIGS. 12(a) (TC) and 12(b) (HDL), using the calibration plots at 182 sec and 154 sec. The slope and intercept from the best line fit of each graph was used to predict the analyte concentration for each current response:

[analyte]=(i_{analyte}−intercept)/slope

The calculated analyte values for TC and HDL cholesterol were used to calculate the concentration of non-HDL cholesterol according to the equation below:

[Non-HDL]=[TC]−[HDL].

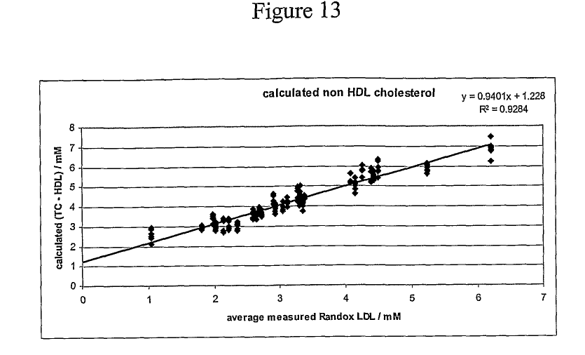

A plot of [non-HDL] (y-axis) vs. [LDL] (x-axis) was made. This graph is shown in FIG. 13. Good correlation between non-HDL cholesterol concentration and LDL cholesterol concentration was observed.

### Conclusions

Electrochemical sensor strips containing total cholesterol and HDL cholesterol sensors can be used to determined plasma non-HDL cholesterol concentration, which gives good correlation to measured values of plasma LDL cholesterol.

The invention has been described with reference to various specific embodiments and examples. However, it is to be understood that the invention is in no way limited to these specific embodiments and examples.

1. A method for the determination of the amount of cholesterol in lipoproteins other than high density lipoproteins in a lipoprotein-containing sample, said method comprising (a) electrochemically determining the amount of cholesterol bound to high density lipoproteins in the sample, (b) electrochemically determining the total amount of cholesterol in the sample, and subtracting the result of (a) from the result of (b).

2. A method according to claim 1, wherein step (a) comprises reacting the sample with a first series of reagents comprising
(a1) a cholesterol ester hydrolysing reagent;
(a2) cholesterol dehydrogenase;
(a3) a coenzyme;
(a4) a redox agent capable of being oxidised or reduced to form a product; and
(a5) a surfactant which selectively breaks down high density lipoproteins,
and electrochemically detecting the amount of product formed.

3. A method according to claim 2, wherein the surfactant (a5) has a differentiation for HDL over LDL given by the equation (i) below of at least 50%:
$$Differentiation ( \% ) = \frac{{G}_{HDL} - {G}_{LDL}}{{G}_{HDL}} \times 100 \\ wherein {G}_{HDL} is \frac{measured ( HDLcholesterol )}{known [ HDLcholesterol ]} , \\ and {G}_{LDL} is \frac{measured ( LDLcholesterol )}{known [ LDLcholesterol ]} . \qquad ( i )$$
wherein measured (HDL cholesterol) or measured (LDL cholesterol) is a measured value relating to the concentration of HDL or LDL respectively.

4. A method according to claim 2, wherein the surfactant (a5) is a sucrose ester, maltoside, hydroxyethylglucamide derivative or N-methyl-N-acyl glucamine derivative.

5. A method according to claim 2, wherein the surfactant (a5) is of formula CH₃(CH₂)₁₃(OCH₂CH₂)_{33.5}OH, a hydroxyethyl glucamide derivative or sucrose monocaprate.

6. A method according to claim 1, wherein step (b) comprises reacting the sample with a second series of reagents comprising
(b1) a cholesterol ester hydrolysing reagent;
(b2) cholesterol dehydrogenase;
(b3) a coenzyme;
(b4) a redox agent capable of being oxidised or reduced to form a product; and optionally
(b5) a surfactant,
and electrochemically detecting the amount of product formed.

7. A method according to claim 6, wherein the surfactant (b5) comprises one or more bile acid derivatives or salts thereof.

8. A method according to claim 6, wherein the sample is reacted substantially simultaneously with the first series of reagents and the second series of reagents.

9. A method according to claim 2, wherein step (a) comprises reacting the sample with the first series of reagents and electrochemically detecting the product formed at a time when the high density lipoproteins preferentially react; and step (b) comprises continuing the reaction between the sample and the first series of reagents and electrochemically detecting the product formed at a time when all lipoproteins react.

10. A method according to claim 2, wherein the first and/or second series of reagents additionally comprises a reductase.

11. A method according to claim 1, wherein the lipoprotein containing sample is whole blood and wherein the method additionally comprises the step of filtering the sample to remove red blood cells.

12. A method according to claim 1, wherein the method is completed within a total period of no more than 3 minutes.

13. A kit for the determination of the amount of cholesterol in lipoproteins other than high density lipoproteins in a lipoprotein-containing sample, the kit comprising
a first electrochemical cell having a working electrode, a reference or pseudo reference electrode and optionally a separate counter electrode;
a first series of reagents comprising
(a1) a cholesterol ester hydrolysing reagent;
(a2) cholesterol dehydrogenase;
(a3) a coenzyme;
(a4) a redox agent capable of being oxidised or reduced to form a product; and
(a5) a surfactant which selectively breaks down high density lipoproteins, said first series of reagents being associated with said first electrochemical cell;
a second electrochemical cell having a working electrode, a reference or pseudo-reference electrode and optionally a separate counter electrode;
a second series of reagents comprising
(b1) a cholesterol ester hydrolysing reagent;
(b2) cholesterol dehydrogenase;
(b3) a coenzyme;
(b4) a redox agent capable of being oxidised or reduced to form a product; and optionally
(b5) a surfactant, said second series of reagents being associated with said second electrochemical cell;
a power supply for applying a potential across each cell;
a measuring instrument for measuring the resulting electrochemical response of each cell; and
a calculator for calculating the amount of cholesterol in lipoproteins other than high density lipoproteins.

14. A kit according to claim 13, wherein the first series of reagents is present in the form of a single first reagent mixture, and the second series of reagents is present in the form of a single second reagent mixture.

15. A kit according to claim 14, wherein each reagent mixture is in dried form.

16. A method of operating a kit, which kit is for the determination of the amount of cholesterol in lipoproteins other than high density lipoproteins in a lipoprotein-containing sample the kit comprising
a first electrochemical cell having a working electrode, a reference or pseudo reference electrode and optionally a separate counter electrode;
a first series of reagents comprising
(a1) a cholesterol ester hydrolysing reagent;
(a2) cholesterol dehydrogenase;
(a3) a coenzyme;
(a4) a redox agent capable of being oxidised or reduced to form a product; and
(a5) a surfactant which selectively breaks down high density lipoproteins, said first series of reagents being associated with said first electrochemical cell;
a second electrochemical cell having a working electrode, a reference or pseudo reference electrode and optionally a separate counter electrode;
a second series of reagents comprising
(b1) a cholesterol ester hydrolysing reagent;
(b2) cholesterol dehydrogenase;
(b3) a coenzyme;
(b4) a redox agent capable of being oxidised or reduced to form a product; and optionally
(b5) a surfactant,
said second series of reagents being associated with said second electrochemical cell;
a power supply for applying a potential across each cell;
a measuring instrument for measuring the resulting electrochemical response of each cell; and
a calculator for calculating the amount of cholesterol in lipoproteins other than high density lipoproteins,
said method comprising
(i) contacting a sample with the first series of reagents, such that the resulting mixture of sample and first series of reagents is in contact with the working electrode of the first electrochemical cell;
(ii) contacting the sample with the second series of reagents, such that the resulting mixture of sample and second series of reagents is in contact with the working electrode of the second electrochemical cell;
(iii) applying a potential across each electrochemical cell;
(iv) electrochemically detecting the amount of product formed in each cell by measuring the resulting electrochemical response; and
(v) calculating the amount of cholesterol bound to non-HDL lipoproteins in the sample.

17. A method according to claim 16, wherein steps (i) and (ii) are carried out substantially simultaneously.

18. A method according to claim 16 wherein step (v) is completed within a period of up to 3 minutes from the time at which the sample is contacted with the first and/or second series of reagents.
